(19) United States
(12) Patent Application Publication
(10) Pub. No.: US 2025/0367467 A1
(43) Pub. Date: Dec. 4, 2025

(54) WEARABLE PHOTOTHERAPY HAIR GROWTH DEVICE

(71) Applicant: SHENZHEN KAIYAN MEDICAL EQUIPMENT CO., LTD, Shenzhen (CN)

(72) Inventors: ALAIN DIJKSTRA, Amstelveen (NL); LI XIANG, Shenzhen (CN); CHEN TONGMING, Shenzhen (CN); YIN ZHICHAO, Shenzhen (CN); Cao Jiaming, Shenzhen (CN)

(73) Assignee: SHENZHEN KAIYAN MEDICAL EQUIPMENT CO., LTD, Shenzhen (CN)

(21) Appl. No.: 19/042,039

(22) Filed: Jan. 31, 2025

(30) Foreign Application Priority Data
May 29, 2024 (CN) 2024212065684
Sep. 5, 2024 (CN) 2024221799210

(51) Int. Cl. A61N 5/06

(52) U.S. Cl. CPC A61N 5/0617; A61N 2005/0647; A61N 2005/0652

(57) ABSTRACT

Embodiments of the present invention provide a wearable phototherapy hair growth device comprising a wearable body adapted to fit the head, a treatment pad configured to emit light to promote hair growth, and a control box for driving the operation of the treatment pad. The treatment pad is detachably connected to the inner surface of the wearable body and the control box is detachably connected to the wearable body and the treatment pad. The device features a simple structure, with the detachable connection between the wearable body and the treatment pad allowing for easy disassembly, enabling convenient cleaning of both components separately.

### TECHNICAL FIELD

The present invention relates generally to the technical field of hair restoration and treatment devices, specifically to a wearable phototherapy device designed to promote hair growth.

### BACKGROUND ART

Hair loss, often caused by factors such as stress, overwork, and genetics, is a common condition that can lead to baldness and negatively impact an individual's appearance and self-esteem. As a result, there is an increasing demand for effective solutions to address hair loss. Among the various treatment methods, phototherapy has been recognized for its potential to stimulate hair growth. Large-scale phototherapy devices, such as laser hair growth instruments, are used in clinical settings for this purpose. However, these devices are expensive, bulky, and not easily accessible to the average consumer due to their high cost. Additionally, their complex structures make them cumbersome to use and often result in suboptimal treatment outcomes, limiting their effectiveness and practicality for regular use.

In response to the growing demand for more convenient and affordable solutions, hair loss treatment products, such as caps designed for phototherapy, have emerged in the market. These caps aim to deliver targeted light therapy to promote hair growth. However, existing options are often complex in design and challenging to clean, leading to issues with user-friendliness and hygiene. Consequently, there remains a need for an improved, user-friendly, and effective device for treating hair loss that addresses the shortcomings of current solutions.

### OBJECTS OF THE INVENTION

Some of the objects of the invention are as follows:
An object of the present invention is to provide a wearable phototherapy device for stimulating hair growth.
Another object of the present invention is to provide a wearable phototherapy device where the treatment pad and the wearable body are detachable and can be cleaned separately.
Another object of the present invention is to provide a wearable phototherapy device where the control box for controlling the functioning of the treatment pad is detachable and can be disassembled to protect the device in the control box from being damaged while cleaning.
Another object of the present invention is to provide a wearable phototherapy device having a treatment pad with cutouts to prevent wrinkles on the treatment pad.
Another object of the present invention is to provide a wearable phototherapy device where the control box is attached to the connecting belt.

### SUMMARY OF THE INVENTION

According to a first aspect of the present invention, a wearable phototherapy hair growth device is provided. The wearable phototherapy hair growth device comprising: a wearable body adapted to fit the head; a treatment pad configured to emit light to promote hair growth; and a control box for driving the operation of the treatment pad; wherein the treatment pad is detachably connected to the inner surface of the wearable body; wherein the side of the treatment pad facing the wearable body is provided with a connecting portion, which extends through the wearable body and is detachably connected to the control box.

In one embodiment of the invention, the treatment pad comprises a light-transmitting layer, a light source layer, and a lining layer; the light-transmitting layer is connected with the lining layer; the light source layer is located between the lining layer and the light-transmitting layer; and a first connector and a second connector are provided on the lining layer.

In one embodiment of the invention, the wearable body comprises a third connector corresponding to the position of the first connector; the third connector is detachably connected with the first connector.

In one embodiment of the invention, the control box comprises a fourth connector corresponding to the position of the second connector; the fourth connector is detachably connected with the second connector and the second connector is arranged on the connecting portion.

In one embodiment of the invention, the first connector is magnetically coupled with the third connector; or the first connector is a hook-and-loop fastener, and the third connector is a Velcro component.

In one embodiment of the invention, the second connector is magnetically coupled with the fourth connector; or the second connector is a first magnetic electrode electrically connected with the light source layer and the fourth connector is a second magnetic electrode that is magnetically coupled with the first magnetic electrode and electrically connected.

In one embodiment of the invention, the light-transmitting layer is a transparent silicone sheet, the lining layer is a silicone sheet, and the light source layer is a flexible LED light board.

In one embodiment of the invention, the light-transmitting layer, the light source layer, and the lining layer are pressed, pasted, and connected by hot pressing or injection molding to form the treatment pad.

In one embodiment of the invention, the treatment pad comprises a plurality of second notches arranged along the length direction of the treatment pad, and the width of the plurality of second notches increases from the bottom to opening direction of each of the plurality of second notches.

In one embodiment of the invention, the control box comprises a housing, an energy storage element, and a control panel mounted within the housing, with the control panel being electrically connected to the energy storage element.

In one embodiment of the invention, the housing includes a box body that accommodates the energy storage element and the inner cavity of the control panel, and a sealing plate covers the box, the sealing plate is detachably connected to the connecting portion.

In one embodiment of the invention, the plurality of second notches are in V-shape and arranged along width of the treatment pad that prevents the treatment pad from wrinkling.

According to a second aspect of the present invention, a wearable phototherapy hair growth device is provided. The wearable phototherapy hair growth device comprising: a wearable body adapted to fit the head of a user; a treatment pad configured to emit light to promote hair growth; a connecting belt for adjusting size of the wearable body, the connecting belt comprises: a control box having an upper casing and a lower casing to provide a housing to accommodate a control panel and an energy storage device; a first end having a first connecting member for connecting to a first side of the wearable body; and a second end having one or more electrodes to electrically connect with the treatment pad and a fourth connector to magnetically connect with a second connector on the treatment pad.

In one embodiment of the invention, one edge of the treatment pad is detachably connected with a connection base and the control box is connected to the connection base.

In one embodiment of the invention, the upper casing and the connection base are provided with a pair of holes to allow the one or more electrodes of the control box to electrically connect with the treatment pad.

In one embodiment of the invention, the treatment pad comprises a light-transmitting layer, a light source layer, and a lining layer; the light-transmitting layer is connected with the lining layer; the light source layer is located between the lining layer and the light-transmitting layer; and a first connector and the second connector are provided on the lining layer.

In one embodiment of the invention, the treatment pad comprises a central pad and a plurality of extension pads radially distributed around the central pad, a primary notch is created by a space between the plurality of extension pads.

In one embodiment of the invention, a secondary notch is provided at the center of each of the plurality of extension pads on each side.

In one embodiment of the invention, the wearable body comprises a connecting port and an edge of the wearable body is provided with a first notch located at the connecting port, and a connecting belt is provided for adjusting the size of the first notch.

In one embodiment of the invention, the control box is detachably attached to the connecting belt by an attachment means that includes but is not limited to a Velcro, a magic buckle, a magic tape, or a magnet.

In the context of the specification, the term “processor” refers to one or more of a microprocessor, a microcontroller, a general-purpose processor, a Field Programmable Gate Array (FPGA) or an Application Specific Integrated Circuit (ASIC), and the like.

In the context of the specification, the phrase “memory unit” refers to volatile storage memory, such as Static Random Access Memory (SRAM) and Dynamic Random Access Memory (DRAM) of types such as Asynchronous DRAM, Synchronous DRAM, Double Data Rate SDRAM, Rambus DRAM, and Cache DRAM, etc.

In the context of the specification, the phrase “storage device” refers to a non-volatile storage memory such as EPROM, EEPROM, flash memory, or the like.

In the context of the specification, the phrase “communication interface” refers to a device or a module enabling direct connectivity via wires and connectors such as USB, HDMI, VGA, or wireless connectivity such as Bluetooth or Wi-Fi, or Local Area Network (LAN) or Wide Area Network (WAN) implemented through TCP/IP, IEEE 802.x, GSM, CDMA, LTE, or other equivalent protocols.

In the context of this specification, terms like “light”, “radiation”, “irradiation”, “emission” and “illumination”, etc. refer to electromagnetic radiation in frequency ranges varying from the visible frequencies to Infrared (IR) frequencies and wavelengths, wherein the range is inclusive of visible light, and IR frequencies and wavelengths. The terms light”, “radiation”, “irradiation”, “emission” and “illumination”, etc. can be originated from light sources such as Light emitting diodes or Laser diodes emitting coherent and non-coherent lights. Preferably, it refers to low-level electromagnetic radiation of low level of red and near-infrared (NIR) light. It is to be noted here that IR radiation can be categorized into several categories according to respective wavelength ranges which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm) and Far IR (15-1000 μm). In this regard, light application is at relatively low energy densities, typically below about 500 mW, as compared to other forms of laser therapy that are used for ablation, cutting, and thermally coagulating tissue. In some instances, the electromagnetic radiation can also be in wavelengths in the blue or ultraviolet regions, especially for treatment of conditions that occur at the skin surface, such as psoriasis or infection.

In the context of the specification, when an element is referred to as being “fixed to” or “disposed to” another element, it may either directly on another element or indirectly on that other element. When a component is said to be “connected” or “connected to” another component, it may be directly connected to another component or indirectly connected to other components on the piece.

In the context of the specification, the terms “first”, “second” and “third” are only used for descriptive purposes and do not implicate the relative importance or to implicitly indicate the quantity of technical features indicated.

In the context of the specification, the term “plurality” means two or more than two, unless otherwise indicated.

In the context of the specification, the term “several” means more than one, unless otherwise specified.

In the context of the specification, “Light Emitting Diodes (LEDs)” refer to semiconductor diodes capable of emitting electromagnetic radiation when supplied with an electric current. The LEDs are characterized by their superior power efficiencies, smaller sizes, rapidity in switching, physical robustness, and longevity when compared with incandescent or fluorescent lamps. In that regard, the one or more LEDs may be through-hole type LEDs (generally used to produce electromagnetic radiations of red, green, yellow, blue and white colors), Surface Mount Technology (SMT) LEDs, Bi-color LEDs, Pulse Width Modulated RGB (Red-Green-Blue) LEDs, and high-power LEDs, etc.

Materials used in the one or more LEDs may vary from one embodiment to another depending upon the frequency of radiation required. Different frequencies can be obtained from LEDs made from pure or doped semiconductor materials. Commonly used semiconductor materials include nitrides of Silicon, Gallium, Aluminum, and Boron, and Zinc Selenide, etc. in pure form or doped with elements such as Aluminum and Indium, etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used for generating a wider color gamut. White and other colored lightings may also be produced using phosphor coatings such as Yttrium Aluminum Garnet (YAG) in combination with a blue LED to generate white light and Magnesium doped potassium fluorosilicate in combination with blue LED to generate red light. Additionally, near Ultraviolet (UV) LEDs may be combined with europium-based phosphors to generate red and blue lights and copper and zinc-doped zinc sulfide-based phosphors to generate green light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety, by reference.

In several embodiments, the one or more LEDs may also be micro-LEDs described through U.S. Pat. Nos. 8,809,126 B2, 8,846,457 B2, 8,852,467 B2, 8,415,879 B2, 8,877,101 B2, 9,018,833 B2 and their respective family members, assigned to NthDegree Technologies Worldwide Inc., which are included herein by reference, in their entirety. The one or more LEDs, in that regard, may be provided as a printable composition of the micro-LEDs, printed on a substrate.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings wherein like reference letters and numerals indicate the corresponding parts in various figures in the accompanying drawings, and in which:

FIG. 1 shows a back view of a wearable phototherapy hair growth device, in accordance with an embodiment of the present invention.

FIG. 2 shows a bottom view of the wearable phototherapy hair growth device in accordance with an embodiment of the present invention.

FIG. 3 shows the structure of a wearable body of the wearable phototherapy hair growth device described in the embodiment of the present application;

FIG. 4 shows the structure of a treatment pad of the wearable phototherapy hair growth device, in an unfolded state, in accordance with an embodiment of the present invention.

FIG. 5 shows an exploded view of the treatment pad of the wearable phototherapy hair growth device in accordance with an embodiment of the present invention.

FIG. 6 illustrates the wearable phototherapy hair growth device with a control box attached to a connecting belt of the wearable phototherapy hair growth device, in accordance with an embodiment of the present invention.

FIG. 7A and FIG. 7B illustrates an exploded view of the control box adaptable to attach on the connecting belt of the wearable phototherapy device in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

The terms “a” and “an” herein do not denote a limitation of quantity, but rather denote the presence of at least one of the referenced items. The terms “having”, “comprising”, “including”, and variations thereof signifies the presence of a component.

In the description of the embodiments of the present application, it is to be understood that the orientations or positional relationships indicated by the terms “length”, “width”, “upper”, “down”, “front”, “rear”, “left”, “right”, “vertical”, “horizontal”, “top”, “bottom”, “inner”, “outer”, etc., are based on the orientations or positional relationships shown in the drawings and are only for the convenience of describing the embodiments of the present application and for simplifying the description, and are not intended to indicate or imply that the device or element referred to must have a particular orientation, be constructed and operated in a particular orientation, Therefore, it cannot be construed as a limitation on the present application.

In addition, the terms “first” and “second” are used for descriptive purposes only and should not be construed as indicating or implying relative importance or implying the number of technical features indicated. Thus, defining the “first” and “second” features may explicitly or implicitly include one or more of these features. In the description of the embodiments of the present application, “plurality” means two or more of them, unless otherwise expressly and specifically qualified.

In the embodiments of the present application, unless otherwise expressly specified and limited, the terms “mounted”, “connected”, “connected”, “fixed”, etc., shall be construed broadly, for example, they may be fixed, detachable, or integral; It can be mechanically or electrically connected; It can be directly connected or indirectly connected through an intermediate medium, it can be the internal connection of two elements or the interaction relationship between two elements. For those of ordinary skill in the art, the specific meaning of the above terms in the embodiments of the present application may be understood according to the specific circumstances.

Embodiments of the present invention disclose a wearable phototherapy device for hair growth addressing the technical issue of current hair loss treatment solutions, where existing hats are complex in structure and difficult to clean. The wearable phototherapy device is a light therapy hat/cap for stimulating hair growth. The wearable phototherapy device comprises a wearable body that fits securely on the head of a user, a therapeutic/treatment pad embedded with light-emitting elements, and a control box for easy operation. The therapeutic/treatment pad emits specific wavelengths of light for stimulating hair follicles and the hat's design ensures that the light is evenly distributed across the scalp. The control box allows the user to adjust settings for light intensity and treatment duration. The therapeutic/treatment pad is designed to detachably connect with the wearable body so that the wearable body can be easily cleaned by removing the therapeutic/treatment pad. The hat/cap is equipped with a connecting port and adjustable straps to accommodate different head sizes, enhancing comfort and usability. The control box is either detachably connected to the therapeutic/treatment pad or is integrated into the adjustable strap of the hat/cap.

Several additional functionalities may also be added to the wearable phototherapy hair growth device. For example, the wearable phototherapy hair growth device may have at least one additional stimulation elements to impart additional therapy to the user. The at least one stimulation element is selected from the group consisting of lasers, heating element, cooling element, vibration element, electrodes, micro-current element. The vibration element cab be used as a massage heads to massage the scalp, or the micro-current element is used to impart micro-current to the skin of the user. The wearable phototherapy device may have one or more sensors to sense the one or more parameters of the skin of the user. Moreover, activation and control of specific stimulation elements may be based upon feedback received from the one or more sensors. The control box is connected to an external device to control the functioning of the wearable phototherapy hair growth device.

Several embodiments of the present invention will now be described in detail with references to FIGS.

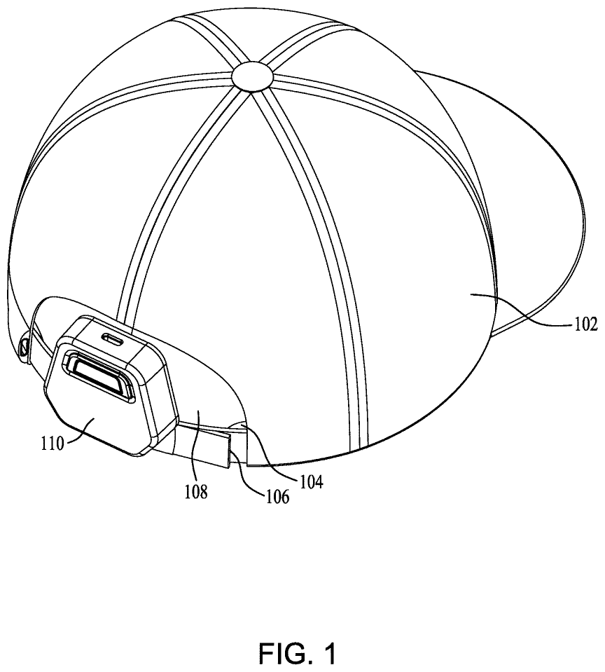

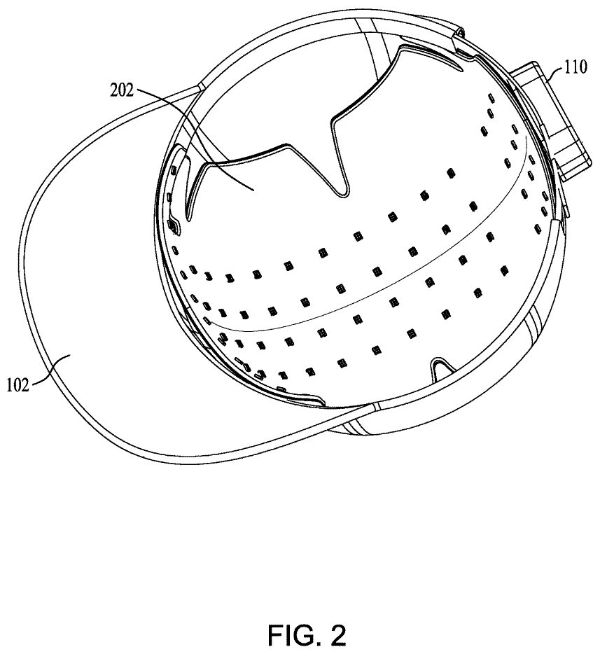

FIG. 1 shows a back view of a wearable phototherapy hair growth device, and FIG. 2 shows a bottom view of the wearable phototherapy hair growth device, described in the embodiment of the present application.

As shown in FIG. 1 and FIG. 2, the embodiment of the present application provides a wearable phototherapy hair growth device, comprising a wearable body 102 for fitting with the head, a treatment pad 202 for emitting light to promote hair growth, and a control box 110 for operating the functioning of the treatment pad. The inner side surface of the treatment pad 202 is detachably connected with the inner side of the wearable body 102. The one side of the treatment pad 202 close to the wearable body 102 is provided with a connecting portion 108, and the connecting portion 108 exposes the wearable body 102. The connecting portion 108 is detachably connected with the control box 110.

The treatment pad 202 is detachably connected with the wearable body 102 so that a user can conveniently disassemble the treatment pad 202 from the wearable body and both can be cleaned separately. The wearable body 102 and the treatment pad 202 are detachably connected with the control box 110 of the wearable phototherapy hair growth device.

The treatment pad 202 is detachably connected with the control box 110 and is exposed on the outer wall surface of the wearable body 102. In the process of cleaning the treatment pad 202 and the wearable body 102, the control box 110 is disassembled from the treatment pad 202, and the device in the control box 110 will not be damaged during the cleaning process. The wearable body 102 can also be worn separately according to the user's needs, such that the wearable body 102 and the treatment pad 202 are used together when providing phototherapy for stimulating hair growth.

In an embodiment of the present application, the wearable body 102 can be selected from various types of headwear, such as a baseball cap or a sunshade cap, making the phototherapy hair growth device a hair growth cap. Alternatively, the wearable body 102 is the shell for a hair growth instrument, such as a hair growth treatment device or a laser hair growth device.

The application provides a wearable phototherapy hair growth device, comprising a wearable body for fitting with the head, a treatment pad for emitting light to promote hair growth, and a control box for driving the work of the treatment pad, wherein the treatment pad is detachably connected with the inner side of the wearable body. The side of the treatment pad close to the wearable body is provided with a connecting part, and the connecting part exposes the wearable body and is detachably connected to the control box. The structure of the wearable phototherapy hair growth device is simple, and the detachable connection between the wearable body and the treatment pad is adopted, so that the treatment pad is convenient to disassemble from the wearable body, and the wearable body and the treatment pad can be cleaned separately. Because the treatment pad is detachably connected with the control box, in the process of cleaning the treatment pad and the wearable body, the control box is disassembled from the treatment pad alone, to protect the device in the control box from being damaged by cleaning the treatment pad.

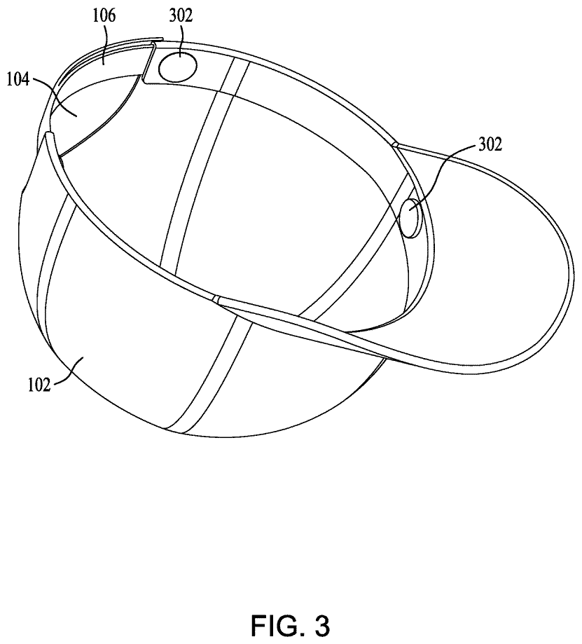

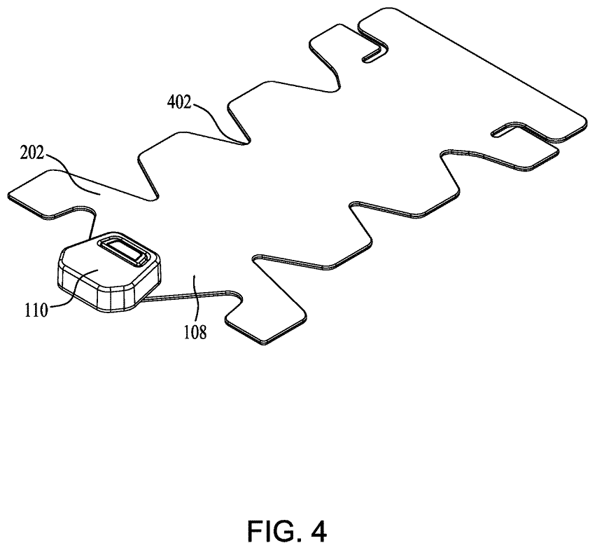

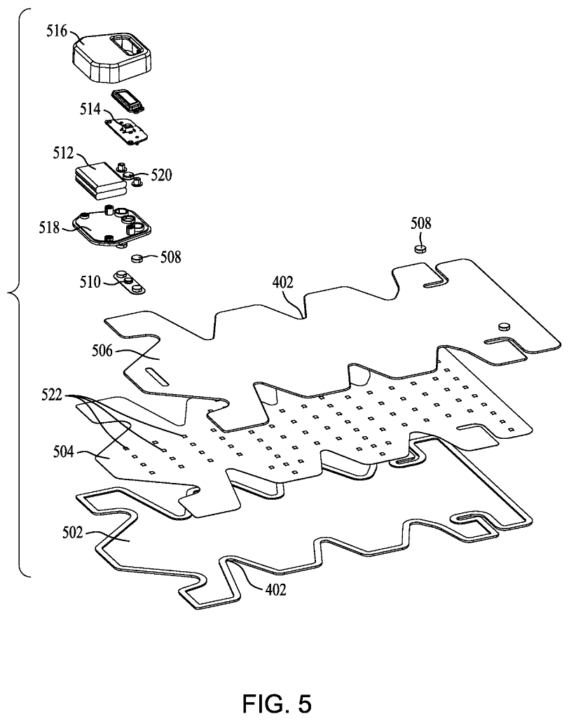

FIG. 3 shows the structure of a wearable body of the wearable phototherapy hair growth device described in the embodiment of the present application; FIG. 4 shows the structure of a treatment pad of the wearable phototherapy hair growth device, in an unfolded state, in accordance with an embodiment of the present invention and FIG. 5 shows an exploded view of the treatment pad of the wearable phototherapy hair growth device in accordance with an embodiment of the present invention.

As shown in FIG. 3 to FIG. 5, the treatment pad 202 comprises a light-transmitting layer 502, a light source layer 504, and a lining layer 506. The light-transmitting layer 502 is connected with the lining layer 506, and the light source layer 504 is located between the lining layer 506 and the light-transmitting layer 502. A first connector 508 and a second connector 510 are installed on the lining layer 506. The wearable body 102 comprises a third connector corresponding to the position of the first connector 508. The third connector 302 is detachably connected with the first connector 508. The control box 110 comprises a fourth connector 520 corresponding to the position of the second connector 510. The fourth connector 520 is detachably connected with the second connector 510, which is arranged on the connecting portion 108.

The light-transmitting layer 502 is made of a transparent silicon sheet, a flexible rubber sheet, or a cloth. The lining layer 506 is made of a transparent silicon sheet, a flexible rubber sheet, or a cloth. The light source layer 504 can be a flexible LED light board. A plurality of light-emitting elements 522 for emitting red light are arranged on the light source layer 504. The light-transmitting layer 502 comprises a plurality of holes to embed the light-emitting elements 522 on the light source layer 504. The lining layer 506 is made of white opaque silica gel material, which is convenient for the light-emitting element 522 on the light source layer 504 to emit red light. The red light has a germination effect required for stimulating the growth of hair follicles. In this embodiment, the light-emitting element 522 may be an LED lamp, a neon lamp, or other lamps that can emit red light. The treatment pad 202 adopts a light-transmitting layer 502 to facilitate its cleaning. Wherein, the light-transmitting layer 502, the light source layer 504, and the lining layer 506 are pressed together to form an integrated treatment pad 202 by hot pressing. In other embodiments, the light-transmitting layer 502, the light source layer 504, and the lining layer 506 may also be pasted and connected or injection molding to form an integrated treatment pad 202.

As shown in FIG. 3 to FIG. 5, the first connector 508 is connected with the third connector 302 magnetically. In an embodiment, the first connector 508 is a magnet, and the third connector 302 is a magnet or an iron block. In another embodiment, the first connector 508 is a Velcro, and the third connector 302 is a Velcro. In another embodiment, the first connector 508 is connected with the third connector 302 in a Snap-On fit, for example, the first connector 508 is a fastener, and the third connector 302 is a buckle position matched with the fastener.

As shown in FIG. 3 to FIG. 5, the second connector 510 is magnetically connected with the fourth connector 520. In an embodiment, the second connector 510 is a magnet, and the fourth connector 520 is also a magnet. In another embodiment, the second connector 510 is a first magnetic suction electrode electrically connected with the light source layer 504, and the fourth connector 520 is a second magnetic suction electrode that is magnetically matched with and electrically connected with the first magnetic suction electrode.

The wearable phototherapy device comprises a plurality of first connectors 508 arranged on the bottom of the lining layer 506 at intervals, and a plurality of third connectors 302 are connected with the plurality of first connectors 508 corresponding to matching setting one-to-one. The wearable phototherapy device comprises a plurality of second connectors 510 arranged on the bottom of the lining layer 506 at intervals, and a plurality of fourth connectors 520 are connected with the plurality of second connectors 510 corresponding to matching settings one-to-one. The first connector 508 is matched with the third connector 302 to realize a detachable connection between the treatment pad 202 and the wearable body 102. The second connector 510 is matched with the fourth connector 520, to realize a detachable connection between the control box 110 and the treatment pad 202, so that the disassembly and cleaning of the treatment pad 202 are convenient. In this embodiment, the fastener may be selected as a button. In this embodiment, if the second connector 510 and the fourth connector 520 are both selected as magnetic suction electrodes, the control box 110 and the treatment pad 202 are magnetically connected and connected through magnetic suction electrodes. If the first connector 508 and the third connector 302 are magnets, the first connector 508, mounted on the lining layer 506, aligns magnetically with the magnet on the treatment pad 202. This magnetic connection between the treatment pad 202 and the inner magnet of the wearable body 102 allows for easy disassembly and cleaning of the treatment pad 202. The first connectors 508 are arranged on the bottom of the lining layer 506.

As shown in FIG. 3, a connecting port 104 corresponding to the position of the connecting portion 108 is formed in the wearable body 102. The edge of the wearable body 102 is provided with a first notch. The wearable body 102 located at the opening side of the connecting port 104 is provided with a connecting belt 106 for adjusting the size of the first notch, and the connecting belt 106 encloses the wearable body 102 to form a connecting port 104.

It should be noted that the connecting belt 106 can be selected to have an elastic belt.

As shown in FIG. 5, a plurality of second notches 402 are formed in the treatment pad 202. The plurality of second notches 402 are arranged along the length direction of the treatment pad 202, and the width of the second notch 402 gradually increases from the bottom of the second notch 402 to the opening direction of the second notch 402.

It should be noted that the second notch 402 can have a V-shape. In this embodiment, three V-shaped second notches 402 are arranged along the width of the light-transmitting layer 502, the light source layer 504, and the lining layer 506 on both sides. This arrangement helps prevent the treatment pad 202 from wrinkling as it bends when inserted into the wearable body 102, ensuring a more comfortable fit for the user when wearing the phototherapy hair growth device.

As shown in FIG. 5, in one embodiment of the present application, the control box 110 comprises a housing and an energy storage element 512, and a control panel 514 is arranged in the housing.

It should be noted that the housing includes a box body 516 containing an energy storage element 512 and an inner chamber for the control panel 514, as well as a sealing plate 518 that covers the box body 516. The sealing plate 518 is detachably connected to the connecting portion 108, and the fourth connector 520 of the control box 110 is mounted on the sealing plate 518. In this embodiment, the energy storage element 512 is a rechargeable battery, which provides power to the control panel 514. The control box 110 further comprises a power button for switching On/Off the light-emitting elements 522. An indicator is provided on the control box 110 to indicate the real-time status of the power button, battery, and/or mode of operation.

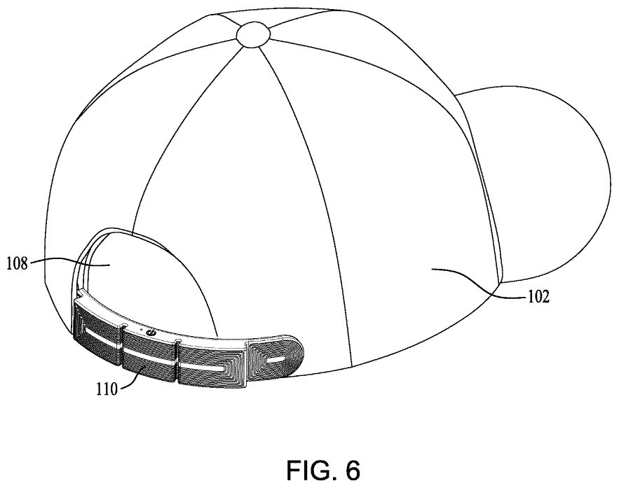

FIG. 6 illustrates the wearable phototherapy hair growth device with a control box attached to a connecting belt of the wearable phototherapy hair growth device, in accordance with an embodiment of the present invention.

As shown in FIG. 6, the connecting port 104 is provided in the wearable body 102 corresponding to the position of the connecting portion 108. The first notch is provided at the edge of the wearable body 102. The connecting belt 106 is provided for adjusting the size of the first notch so that the cap can be adjusted according to the user's head size. The control box 110 is attached or fixed over the connecting belt 106. The shape of the control box 110 is strip-shaped to conform to the shape of the connecting belt 106. The control box 110 is attached to the connecting belt 106 using a hook and loop fastener or Velcro. The fourth connector 520 on the control box 110 is magnetically and electrically connected with the second connector 510. The connection between the second connector and the fourth connector ensures the control and operation of light-emitting elements 522 in the light source layer 504 by the control box 110.

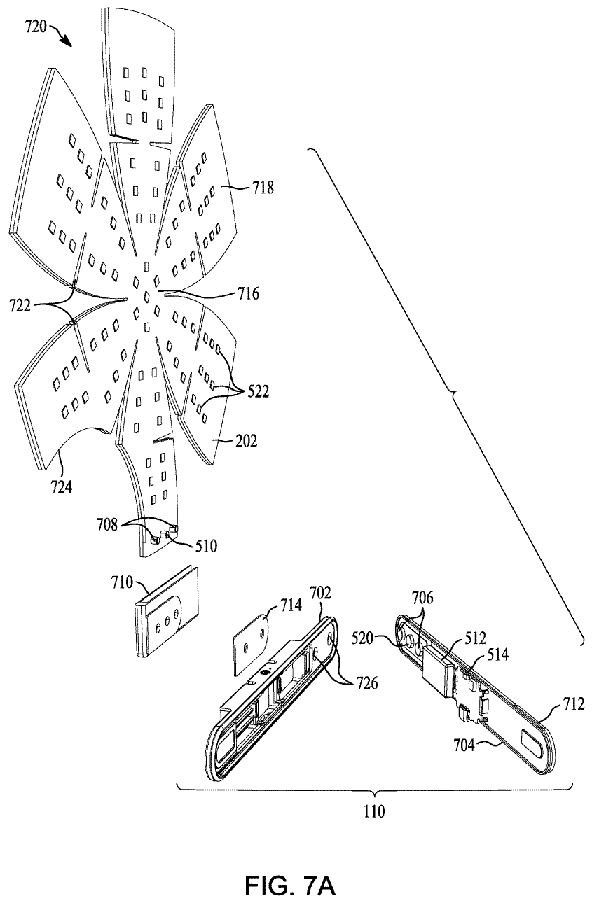

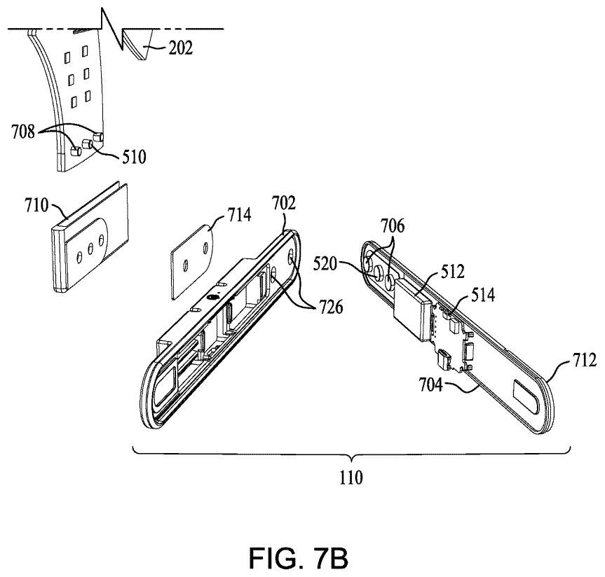

Referring to FIGS. 7A and 7B, an exploded view of control box 110 that is adaptable to attach to the connecting belt of the wearable device is shown. In some embodiments, the control box 110 can also serve the purpose of the connecting belt to hold the wearable device over the head of a user. The edge of the treatment pad 202 is detachably connected with a connection base 710. The connection base 710 includes two parallel and spaced apart connecting piece. One of the connecting piece is fitted with the treatment pad 202. The control box 110 is located at the connection base 710 at an end away from the treatment pad 202. The treatment pad 202 is provided with the second connector 510, one end of which is inserted in the connection base 710. The second connector 510 provided with the connection base 710 connects with the fourth connector 520. The connection base 710 may be detachably connected to the wearable body 102, and the treatment pad 202 is detachably connected to the connection base 710.

In an embodiment of the present invention, an alternate configuration of treatment pad 202 is provided. The treatment pad 202 comprises a central pad 716 and a plurality of extension pads 718 radially distributed around the central pad 716, such that there is a gap between two adjacent extension pads 718. The gap between adjacent extension pads 718 creates a primary notch 720 and the width of the primary notch 720 gradually increases in the direction away from the central pad 716. A secondary notch 722 is provided on the side of extension pads 718 facing the primary notch 720. The secondary notch 722 is provided at the center in the length direction of each of the extension pads on each side. The secondary notch 722 is provided on both sides of the extension pads 718, such that on a first side, the secondary notch 722 faces one primary notch 720, and on the other side, the secondary notch 722 faces another primary notch 720. The primary notch 720 reduces the possibility of stacking between adjacent extension pads 718 and the secondary notch 722 prevents any extension pads 718 from generating wrinkles along the length direction, thereby improving the wearing comfort of the user. One side of the two extension pads 718 of the treatment pad 202 are cut in arch shape to form a relief notch 724 so as to conform to the shape of the wearable body 102 corresponding to the position of the first notch on the wearable body 102.

The control box 110 is elongated rectangular in shape to fasten one end of the wearable device with the other end. The control box 110 comprises an upper casing 702 and a lower casing 704 configured to fit with each other. The upper casing 702 and the lower casing 704 provide a housing in between to accommodate the energy storage element 512 and the control panel 514. The control box 110 comprises one or more electrode pins 706 at a first end, and a second end 712 of the control box 110 is provided with a first connecting member that connects to a second connecting member provided on the wearable body 102. The first connecting member is detachably connected with the second connecting member to secure or attach the control box 110 on the wearable phototherapy device. The attachment means include a Velcro, magnet, a fastener, or a loop-and-hook mechanism. The number of the first connecting member and the second connecting member is not limited and can be determined according to actual need.

In an embodiment, the first connector is magnetically attached with the second connecting member. The first connecting member and the second connecting member are both magnets, or one of the first connecting member and the second connecting member is a magnet and other is an iron block. In an alternate configuration, the first connecting member is a magic buckle, and the second connecting member is a magic tape. Alternatively, the first connecting member is snap-connected with the second connecting member. In another alternate embodiment, the first connecting member and the second connecting member can also be detachably connected by fasteners, such as screws.

At the first end of the control box 110, a fourth connector 520 is provided in the control box 110 between the one or more electrode pins 706. The treatment pad 202 having a plurality of light-emitting elements 522 comprises one or more electrode pins 708 that connect with the one or more electrode pins 706 to provide an electrical connection between the treatment pad 202 and the control box 110. The second connector 510 is provided between the one or more electrode pins 708 of the treatment pad 202. The second connector 510 and the fourth connector 520 provide a magnetic connection between the treatment pad 202 and the control box 110 so that the treatment pad 202 gets firmly coupled to the control box 110. A protective element 714 shields the one or more electrode pins 706 and the fourth connector 520 in the control box 110 from any damage. The protective element and the upper casing 702 of the control box are provided with two holes 726 for allowing the one or more electrode pins 708 of the treatment pad 202 to pass through and connect with the one or more electrode pins 706 of the control box 110. The upper casing has a power button for switching On/Off the light-emitting elements 522. An indicator is provided adjacent to the power button to indicate the real-time status of the power button. The control box 110 is located at the opening side of the connecting port 104 and is used to adjust the size of the first notch of the wearable body 102.

In another embodiment of the present invention, the connecting port 104 is provided on the wearable body 102 and the first notch is located on the opening side of the connecting port 104 for adjusting the size of the first notch. The connecting belt 106 is of the same size as the first notch, and the connecting belt 106 and the wearable body 102 form the connecting port 104. The control box 110 is provided on the connecting belt 106. The connecting belt 106 can be used to adjust the circumference of the edge of the wearable body 102 to fit different users. The control box 110 is arranged on the outer side of the connecting belt 106 to improve the wearing comfort. In this configuration, the control box 110 is detachably attached to the connecting belt 106. The control box 110 and the connecting belt 106 can be magnetically matched, or one is provided with a magic buckle and the other is provided with a magic tape, or one is provided with a Velcro buckle and the other is a Velcro.

In an embodiment of the present invention, the treatment pad 202 is provided with one or more stimulation elements to provide alternate therapy in addition to phototherapy to a user wearing the wearable phototherapy device. The one or more stimulation elements are selected from a group consisting of but not limited to a vibration element, a heating element, a cooling element, and a transducer element. For example, the treatment pad 202 comprises one or more massage heads to provide massage to the scalp of a user that nourishes hair follicles and stimulates hair growth.

In an embodiment of the present invention, the device is configured to connect with an external communication device over a communication interface. The control box 110 includes a processor, a memory unit, and a communication interface. The communication with the external communication device is routed through the communication interface. The control box 110 is configured to connect with the external communication device and receives an input. The input may correspond to activating the light-emitting elements 522 for phototherapy or controlling one or more parameters of operating the light-emitting elements 522. The external communication device may be a smartphone, a desktop computer, a tablet, a Personal Digital Assistant (PDA), a smartwatch, or the like.

In an embodiment of the present invention, the wearable phototherapy device comprises at least one stimulation element to provided additional therapy to the user. The at least one stimulation element is selected from a group consisting of lasers, heating element, cooling element, vibration element, electrodes, micro-current element and a combination thereof. The micro-current element provides micro-current therapy to the user, the vibration element provides massage therapy and the heating element provide heat therapy. The micro-current element or electrodes are either provided in the treatment pad or are provided on the wearable device.

In an embodiment of the present invention, the wearable device further comprises one or more sensor to sense one or more parameters of the skin. The wearable device may communicates one or more parameters of the skin to the external communication device. The wearable device on sensing the one or more parameters of the skin may regulate the operation of one or more stimulation element.

Various modifications to these embodiments are apparent to those skilled in the art, from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A wearable phototherapy hair growth device, comprising:
a wearable body adapted to fit head of a user;
a treatment pad configured to emit light to promote hair growth;
a control box for controlling functioning of the treatment pad;
wherein the treatment pad is detachably connected to an inner surface of the wearable body; and
wherein a first end of the treatment pad facing the wearable body is provided with a connecting portion, which extends through the wearable body and is detachably connected to the control box.

2. The wearable phototherapy hair growth device of claim 1, wherein the treatment pad comprises a light-transmitting layer, a light source layer, and a lining layer; the light-transmitting layer is connected with the lining layer; the light source layer is located between the lining layer and the light-transmitting layer; and a first connector and a second connector are provided on the lining layer.

3. The wearable phototherapy hair growth device of claim 2, wherein the wearable body comprises a third connector corresponding to a position of the first connector; and the third connector is detachably connected with the first connector.

4. The wearable phototherapy hair growth device of claim 2, wherein the control box comprises a fourth connector corresponding to a position of the second connector; and the fourth connector is detachably connected with the second connector and the second connector is arranged on the connecting portion.

5. The wearable phototherapy hair growth device of claim 3, wherein the first connector is magnetically coupled with the third connector; or the first connector is a hook-and-loop fastener, and the third connector is a Velcro component.

6. The wearable phototherapy hair growth device of claim 4, wherein the second connector is magnetically coupled with the fourth connector; or the second connector is a first magnetic electrode electrically connected with the light source layer and the fourth connector is a second magnetic electrode that is magnetically coupled with the first magnetic electrode and electrically connected.

7. The wearable phototherapy hair growth device of claim 2, wherein the light-transmitting layer is a transparent silicone sheet, the lining layer is a silicone sheet, and the light source layer is a flexible LED light board.

8. The wearable phototherapy hair growth device of claim 2, wherein the light-transmitting layer, the light source layer, and the lining layer are pressed, pasted, and connected by hot pressing or injection molding to form the treatment pad.

9. The wearable phototherapy hair growth device of claim 1, wherein the treatment pad comprises a plurality of second notches arranged along length of the treatment pad, and width of the plurality of second notches increases from bottom to opening direction of each of the plurality of second notches.

10. The wearable phototherapy hair growth device of claim 1 further comprising at least one stimulation element selected from a group consisting of Light Emitting diodes (LEDs), lasers, heating element, cooling element, vibration element, electrodes, micro-current element and a combination thereof.

11. The wearable phototherapy hair growth device of claim 1, wherein the control box comprises a housing, an energy storage element, and a control panel mounted within the housing, with the control panel being electrically connected to the energy storage element.

12. The wearable phototherapy hair growth device of claim 11, wherein the housing includes a box body that accommodates the energy storage element and an inner cavity of the control panel, and a sealing plate covers the box body, the sealing plate is detachably connected to the connecting portion.

13. A wearable phototherapy hair growth device, comprising:
a wearable body adapted to fit the head of a user;
a treatment pad configured to emit light to promote hair growth;
a connecting belt for adjusting the size of the wearable body, the connecting belt comprises:
a control box having an upper casing and a lower casing to provide a housing to accommodate a control panel and an energy storage device;
a first end having a first connecting member for connecting to a first side of the wearable body; and
a second end having one or more electrodes to electrically connect with the treatment pad and a fourth connector to magnetically connect with a second connector on the treatment pad.

14. The wearable phototherapy hair growth device of claim 13, wherein one edge of the treatment pad is detachably connected with a connection base and the control box is connected to the connection base.

15. The wearable phototherapy hair growth device of claim 14, wherein the upper casing and the connection base are provided with a pair of holes to allow the one or more electrodes of the control box to electrically connect with the treatment pad.

16. The wearable phototherapy hair growth device of claim 13, wherein the treatment pad comprises a light-transmitting layer, a light source layer, and a lining layer; the light-transmitting layer is connected with the lining layer; the light source layer is located between the lining layer and the light-transmitting layer; and a first connector and the second connector are provided on the lining layer.

17. The wearable phototherapy hair growth device of claim 16, wherein the treatment pad comprises a central pad and a plurality of extension pads radially distributed around the central pad, a primary notch is created by a space between the plurality of extension pads.

18. The wearable phototherapy hair growth device of claim 17, wherein a secondary notch is provided at the center of each of the plurality of extension pads on each side.

19. The wearable phototherapy hair growth device of claim 13, wherein the wearable body comprises a connecting port and an edge of the wearable body is provided with a first notch located at the connecting port, and a connecting belt is provided for adjusting the size of the first notch.

20. The wearable phototherapy hair growth device of claim 19, wherein the control box is detachably attached to the connecting belt by an attachment means that includes but is not limited to a Velcro, a magic buckle, a magic tape, or a magnet.
